(19) United States
(12) Patent Application Publication
(10) Pub. No.: US 2025/0116556 A1
(43) Pub. Date: Apr. 10, 2025

(54) TEMPERATURE PROBE INSERT, ASSEMBLY THEREOF, AND KIT THEREOF

(71) Applicant: ENTEGRIS, INC., Billerica, MA (US)

(72) Inventors: Jacob Peabody, Roxbury, MA (US); Vy Tran, Ho Chi Minh City (VN)

(21) Appl. No.: 18/907,434

(22) Filed: Oct. 4, 2024

Related U.S. Application Data
(60) Provisional application No. 63/543,016, filed on Oct. 6, 2023.

(51) Int. Cl. G01K 1/14; B65D 79/02

(52) U.S. Cl. CPC G01K 1/14; B65D 79/02; B65D 2201/00

(57) ABSTRACT

A temperature probe insert includes a coupling portion, a first arm extending from the coupling portion, and a second arm extending from the coupling portion. The first arm and the second arm extend from different sides of the coupling portion. The coupling portion includes an aperture that extends through the coupling portion. The first arm and the second arm are configured to contact a storage bag shell and prevent movement of the coupling portion in the opening in the storage bag shell. The aperture is configured to direct a temperature probe into a storage bag disposed in the internal volume of the storage bag shell. A storage bag temperature monitoring assembly includes a temperature probe insert and a storage bag shell. A kit includes a temperature probe insert and a temperature probe.

### FIELD

This disclosure relates to an insert for a temperature probe. More specifically, this disclosure relates to an insert for aligning a temperature probe in a storage bag shell.

### BACKGROUND

Storage bags can be employed to store a variety of fluids. Storage bags can be used to storage fluids at a desired temperature. Storage bag shells can be used to safely contain and hold a storage bag during storage. Further, storage bag shells can be used to protect storage bags during heating, cooling, or both heating and cooling of the fluid contained within the storage bag. For example, a storage shell can contain and hold a storage bag while the fluid contained therein is being frozen. For example, a storage shell can contain and hold a storage bag while the frozen fluid contained therein is thawed. In one example, such storage bags may contain biological fluids that undergo controlled freezing and thawing.

### SUMMARY

In an embodiment, a temperature probe insert includes a coupling portion, a first arm extending from the coupling portion, and a second arm extending from the coupling portion. The coupling portion includes an aperture that extends through the coupling portion. The first arm and the second arm extend from different sides of the coupling portion. The temperature probe insert is configured to be removably disposed in an opening in a storage bag shell and the first arm and the second arm are configured to contact the storage bag shell to prevent movement of the coupling portion in the opening in the storage bag shell. The aperture configured to direct a temperature probe into a storage bag disposed in the internal volume of the storage bag shell.

In an embodiment, a storage bag temperature monitoring assembly includes a storage bag shell and a temperature probe insert. The storage bag shell includes a top and a base top disposed on the base to form an internal volume configured contain a storage bag. The storage bag shell including an opening. The temperature probe insert is removably disposed in the opening in the storage bad shell. The temperature probe insert includes a coupling portion, a first arm extending from the coupling portion, and a second arm extending from the coupling portion. The coupling portion includes an aperture that extends through the coupling portion. The first arm and the second arm extend from different sides of the coupling portion. The first arm and the second arm contact the storage bag shell to prevent movement of the coupling portion in the opening in the storage bag shell. The aperture is configured to direct a temperature probe into the storage bag disposed in the internal volume of the storage bag shell.

In an embodiment, a kit includes a temperature probe insert and a temperature probe. The temperature probe insert includes a coupling portion, a first arm extending from the coupling portion, and a second arm extending from the coupling portion. The coupling portion includes an aperture that extends through the coupling portion. The first arm and the second arm extend from different sides of the coupling portion. The temperature probe insert is configured to be removably disposed in an opening in a storage bag shell, and the first arm and the second arm are configured to contact the storage bag shell to prevent movement of the coupling portion in the opening in the storage bag shell. The temperature probe is configured to be disposed in the aperture of the temperature probe insert. The aperture is configured to direct the temperature probe into a storage bag disposed in the internal volume of the storage bag shell with the temperature probe being configured to detect a temperature of the fluid in the storage bag.

### BRIEF DESCRIPTION OF DRAWINGS

FIG. 1 is a front perspective view of an embodiment of a storage bag temperature monitoring assembly.

FIG. 2 is a front perspective view of a storage bag shell of the storage bag temperature monitoring assembly in FIG. 1, according to an embodiment.

FIG. 3 is an exploded view of the storage bag temperature monitoring assembly in FIG. 1, according to an embodiment.

FIG. 4 is a cross-sectional view of a temperature probe insert of the storage bag temperature monitoring assembly in FIG. 1, according to an embodiment.

FIG. 5 is a cross-sectional view of the storage bag temperature monitoring assembly in FIG. 1 in which a temperature probe is omitted, according to an embodiment

FIG. 6 is a front perspective view of an embodiment of a storage bag temperature monitoring assembly.

FIG. 7 is an exploded view of the storage bag temperature monitoring assembly in FIG. 6, according to an embodiment.

FIG. 8 is a partial cross-sectional view of the storage bag temperature monitoring assembly in FIG. 6, according to an embodiment.

FIG. 9 is a partial front perspective view of the storage bag temperature monitoring assembly in FIG. 6 with the temperature probe omitted, according to an embodiment.

FIG. 10 is a bottom perspective view of a temperature probe insert of the storage bag temperature monitoring assembly in FIG. 6, according to an embodiment.

Like numbers represent like features.

### DETAILED DESCRIPTION

This disclosure relates to an insert for a temperature probe. More specifically, this disclosure relates to an insert for aligning a temperature probe in a storage bag shell.

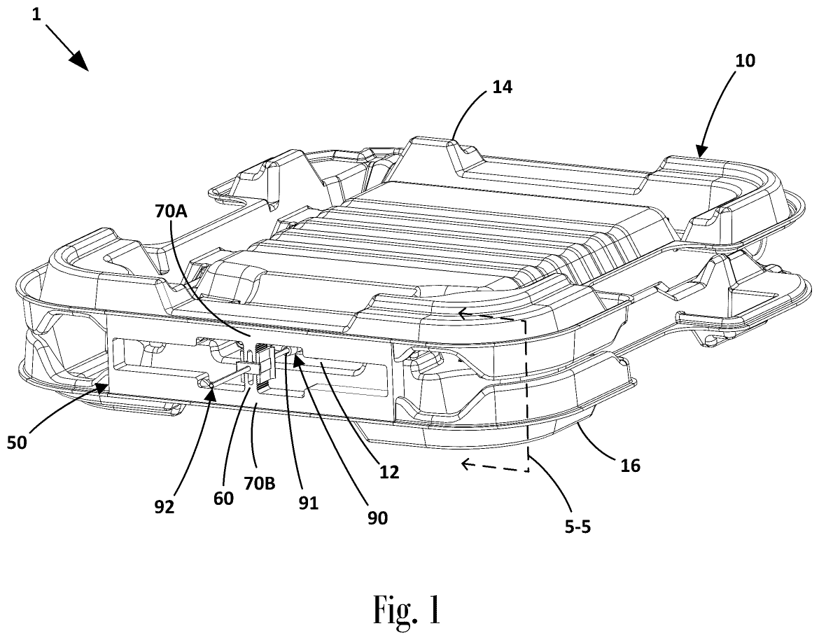

FIG. 1 shows a front perspective view of an embodiment of a storage bag temperature monitoring assembly 1. The assembly 1 includes a storage bag shell 10 and a temperature probe insert 50. The storage bag shell 10 is configured to contain a storage bag 90. The storage bag 90 can be a liquid storage bag configured to contain a liquid. The liquid may be frozen. In an embodiment, the storage bag 90 is a biological storage bag configured to contain a biological fluid. In such an embodiment, the assembly 1 can be configured to hold the storage bag 90 while the liquid within the storage bag 90 is at least one of frozen (e.g., cooled to at or below the freezing temperature of the liquid) and thawed (e.g., heated to above the freezing temperature of the liquid).

The storage bag shell 10 includes an internal volume 12. The storage bag 90 is disposed in the internal volume 12 of the storage bag shell 10. The storage bag 90 is made of a flexible polymeric material (e.g., a polymer, a co-polymer, and the like). For example, the storage bag 90 is made of a polymeric material capable of operating under a temperature change from 40° C. to −85° C. The flexible polymeric material may be, for example but not limited to, polyethylene or the like. In an embodiment, the storage bag 90 can be formed as discussed in U.S. Patent Application Publication No. 2018/0021218, which is incorporated by reference. The storage bag shell 10 is made of a rigid material (e.g., a rigid polymeric material). For example, the rigid polymeric material of the storage bag 10 is a polymeric material capable of operating under a temperature variation from 40° C. to −85° C. The rigid polymeric material be, for example but not limited to, polycarbonate, fluoropolymer, high-density polyethylene (“HDPE”), polycarbonate, a combination thereof, or the like. In an embodiment, the storage bag shell 10 can be formed as discussed in one or more of U.S. Patent Application Publication No. 2022/0298462 and U.S. Patent Application Publication No. 2022/029578, which are incorporated by reference. The storage bag shell 10 is configured to provide protection to the storage bag 90. The storage bag shell 10 is a rigid shell configured to protect a storage bag 90 disposed in the internal volume 12.

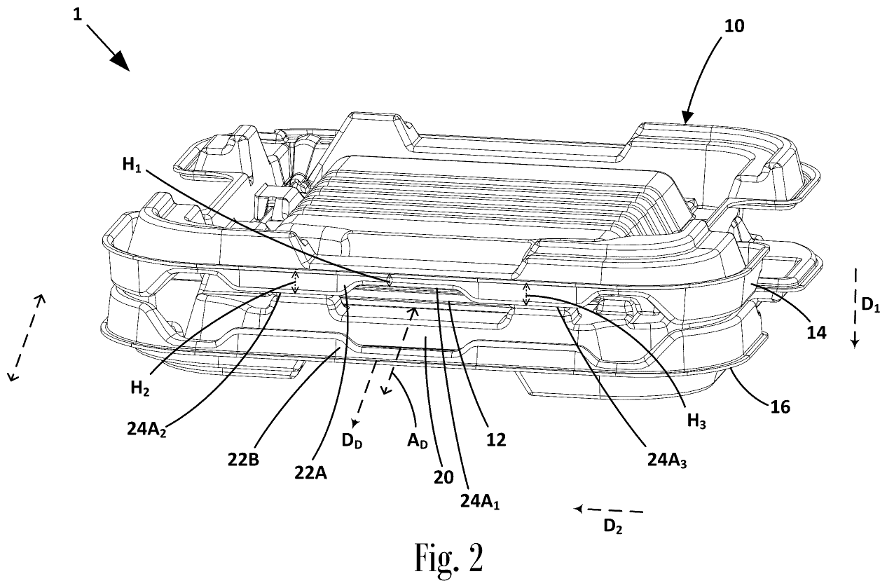

The storage bag shell 10 includes a top 14 and a base 16. The top 14 and the base 16 define the internal volume 12 of the storage bag shell 10. For example, the internal volume 12 is formed between the top 14 and the base 16. For example, the storage bag 90 is stored in the assembly 1 by placing the storage bag the storage bag shell 10 into the portion of the internal volume formed by the base 16 and then placing the top 14 onto the base 16. The storage bag 90 is then trapped within the internal volume 12. In an embodiment, the storage bag shell 10 may include one or more pins 11 (Shown in FIG. 2) that are slid into aligned openings in the top 14 and the base 16, when the top 14 is disposed on the base 16. The inserted pin(s) 11 can be configured to prevent accidental separation of the top 14 and the base 16 from each other.

FIG. 2 is a front perspective view of the assembly 1 in which the insert 50 and the temperature probe 92 are omitted. The storage bag shell 10 includes an opening 20. As shown in FIG. 1 The temperature probe insert 50 is removably disposed in the opening 20 in the storage bag shell 10.

The temperature probe insert 50 is configured to align a temperature probe 92 within the storage bag 90. The temperature probe insert 50 includes an aperture 52 for directing the temperature probe 92. The aperture 52 of the insert 50 is configured to direct the temperature probe 92 into a storage bag 90 disposed in the internal volume 12. The aperture 52 is discussed in more detail below.

Referring back of FIG. 2, in the illustrated embodiment, the opening 20 is defined by the top 14 and the base 16. For example, the opening 20 is an opening formed between the top 14 and the base 16 (e.g., formed by a lower surface of the top 14 and an upper surface of the base 16). The opening 20 extends into the interior of the storage bag shell 10. For example, the opening 20 extends to the internal volume 12. The temperature probe insert 50 is placed between the top 14 and the base 16 before the top 14 is disposed on the base 16. The placement of the top 14 on the base 16 is configured to traps the insert 50 within the opening 20. The insert 50 may be removed upon displacement of the top 14 from the base 16 (e.g., lifting of the top 14 from the base 16). The insert 50 The disposition of the insert 50 in the opening is discussed in more detail below.

The storage bag shell 10 may include a first ridge 22A and a second ridge 22B that each extend into the opening 20 (e.g., extend perpendicular to the axis A_{D} of the opening 20). The top 14 includes the first ridge 22A. The base 16 includes the second ridge 22B. Accordingly, the first ridge 22A may be referred to as the upper ridge, and the second ridge 22B may be referred to as the lower ridge.

As shown in FIG. 2, the base 16 includes the second ridge 22B. The second ridge 22B may respectively have features as similarly discussed with respect to the first ridge 22A. In an embodiment, the storage bag shell 10 may include just one of the first ridge 22A and the second ridge 22B.

As shown in FIG. 2, the first ridge 22A includes a plurality of ridge portions 24A₁, 24A₂, 24A₃. In the illustrated embodiment, the ridge portions include a first ridge portion 24A₁, a second ridge portion 24A₂, and a third ridge portion 24A₃. The first ridge portion 24A₁ is disposed between the second ridge portion 24A₂ and the third ridge portion 24A₃. For example, the second ridge portion 24A₂ and the third ridge portion 24A₃ can be opposite end portions of the first ridge 22A. The first ridge portion 24A₁ can be referred to as a middle ridge portion, the second ridge portion 24A₂ can be referred to as the first end ridge portion, and the third ridge portion 24A₂ can be referred to as the second end ridge portion.

The ridge portions 24A₁, 24A₂, 24A₃ have a plurality of heights h₁, h₂, h₃. The height of a ridge portion is a length of said ridge portion that extends into the opening (e.g., height measured in vertical direction D₁). The middle ridge portion 24A₁ has a first height h₁, the first end ridge portion 24A₂ has a first height h₂, and the third end ridge portion 24A₃ has a third height h₃. In the illustrated embodiment, the ridge portions 24A₁, 24A₂, 24A₃ have two different heights (e.g., height h₂ is the same as height H₃). The end ridge portions 24A₂, 24A₃ have same height (e.g., height h₂/h₃) that is different from height h₁ of the middle ridge portion 24A₁. The end ridge 24A₂, 24A₃ portions each have a respective height h₂, h₃ is longer than the height h₁ of the middle ridge portion 24A₁ (i.e., height h₂>height h₁ and height h₃>height h₁). In another embodiment, the end ridge 24A₂, 24A₃ portions may each have a respective height h₂, h₃ that is shorter than the height h₁ of the middle ridge portion 24A₁ (i.e., height h₂<height h₁ and height h₃<height h₁).

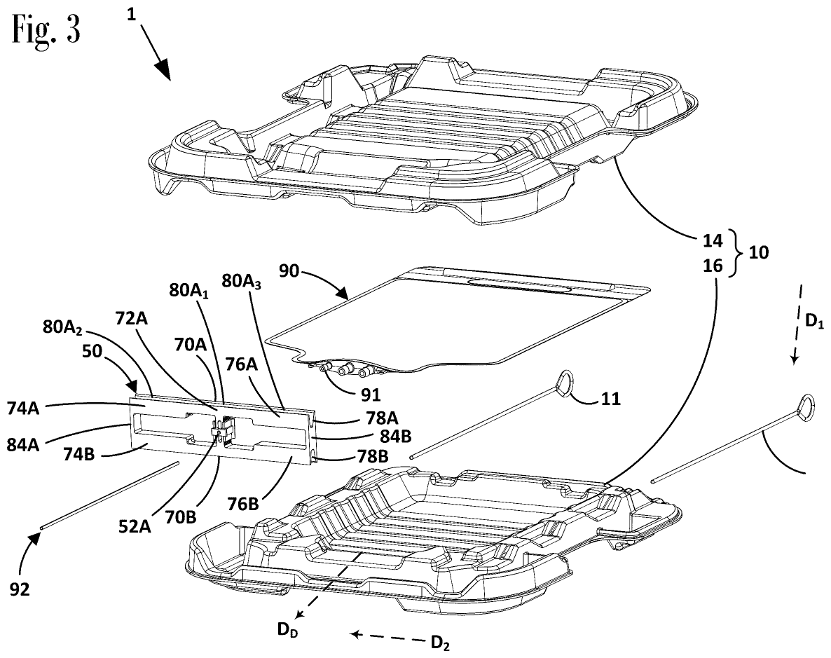

FIG. 3 is an exploded view of the assembly 1, according to an embodiment. The insert 50 includes a coupling portion 60 and a plurality of arms 70A, 70B that each extend from the coupling portion 60. The aperture 52 extends through the coupling portion 60 of the insert 50. The coupling portion 60 of the insert 50 is disposed in the opening 20 of the storage bag shell 10.

The aperture 52 is configured to direct a temperature probe 92 into the storage bag 90. The aperture 52 has an outlet 54 that faces towards a central portion of the internal volume 12. For example, the central portion of the internal volume 12 is the portion of the internal volume that is a middle portion of the internal volume 12 along each major axis (e.g., the X axis, Y axis, and the Z axis). In one embodiment, the central portion of the internal volume is the middle 50% of the internal volume along the X axis (e.g., 25% to 75% of the length along the X axis), the middle 50% of the internal volume 12 along the Y axis (e.g., 25% to 75% of the length along the Y axis), and the middle 50% of the internal volume 12 along the Z axis (e.g., 25% to 75% of the length along the Z axis). In one embodiment, the central portion of the internal volume is the middle 30% of the internal volume along the X axis (e.g., 35% to 65% of the length along the X axis), the middle 50% of the internal volume 12 along the Y axis (e.g., 35% to 65% of the length along the Y axis), and the middle 50% of the internal volume 12 along the Z axis (e.g., 35% to 65% of the length along the Z axis).

As shown in FIG. 1, the aperture 52 directs the probe 92 into a port 91 of the storage bag 90. The insert 50 is configured to direct the probe 92 such that an end 94 of the probe 92 is disposed within the storage bag 90. For example, the end 94 of the probe 92 can be configured to be in contact with the fluid within the storage bag 90.

The aperture 52 has a size configured to provide a fit between the aperture 52 and the probe 92. In an embodiment, the fit may be a clearance fit that allows for some movement of the probe 92 within the aperture 52. The fit of the aperture 52 is configured to prevent the probe 92 from extending more than 3° from aligned (with the aperture 52). The fit of the aperture 52 is configured to prevent the probe 92 from extending more than 1° from aligned (with the aperture 52).

The aperture 52 can be configured to be adjustable within the coupling portion 60. For example, a vertical positon of the aperture 52 in the coupling portion 60 is adjustable (e.g., adjustable in direction D₁). The adjustment of the aperture 52 may be configured to allow for adjusting the vertical position of the aperture 52 such that the aperture 52 directs/holds the temperature probe 92 at the desired location (e.g., height) within the bag 92. In another embodiment, the aperture 52 may not be adjustable within the coupling portion 60.

The plurality of arms 70A, 70B are configured to contact the storage bag shell 10 to prevent perpendicular movement of the coupling portion 60 within the opening 20 (e.g., movement perpendicular to opening, movement periocular to the depth direction D_{D} of the opening 20). For example, the plurality of arms 70A, 70B contact the storage bag shell 12 preventing movement along a first perpendicular direction D₁ (e.g., in the vertical direction) and along a second perpendicular direction D₂ (e.g., in the horizontal direction).

As shown in FIG. 3, the plurality of arms 70A, 70B include a first arm 70A and a second arm 70B. The first arm 70A and the second arm 70B extend from different sides of the coupling portion 60. The first arm 70A and the second arm 70B extend from opposite sides of the coupling portion 60A. For example, the first arm 70A and the second arm 70B each extend perpendicular to the aperture 52 (e.g., first arm 70A extending in direction D₂, second arm 70B extending in a direction opposite to direction D₁).

The first arm 70A includes an intermediate portion 72A, a first branch 76A, and a second branch 76A. The intermediate portion 72A extends from the coupling portion 60. The first branch 76A and the second branch 76A extend from opposite sides of the intermediate portion 72A. For example, the intermediate portion 72A connects each of the first and second branches 76A, 76B to the coupling portion 60A. As shown in FIG. 3, the intermediate portion 72A, the first branch 74A, and the second branch 76A form a substantially T-shaped member (e.g., form a T shape, form a Y shape, form a \hat{ } shape, or the like). It should be appreciated that the second arm 70B may have features as similarly discussed herein for the first arm 70A (e.g., an intermediate portion, a first branch, a second branch, etc.).

The first arm 70A of the insert 50 includes a (first) groove 78A. The first groove 78A is formed in a top side of the first arm 70A (e.g., in the top of the insert 50). The first groove 78A is configured to receive the first ridge 22A of the storage bag shell 10. The second arm 70B of the insert 50 includes a (second) groove 78B. The second groove 78B is formed in a bottom side of the second arm 70B (e.g., in the bottom of the insert 50). The second groove 78B is configured to receive the second ridge 22B of the storage bag shell 10.

The engagement of the grooves 78A, 78B of the insert 50 with the ridges 22A, 22B of the storage bag shell 10 is configured to prevent movement of the insert 50 relative to the storage bag shell 10. For example, the engagement of the grooves 78A, 78B and the ridges 22A, 22B prevents removal of the insert 50 from the opening 20 while the top 14 is disposed on the base 16. For example, the engagement of the grooves 78A, 78B of the insert 50 with the ridges 22A, 22B of the storage bag shell 10 prevents the insert 50 from being removed from the opening 20, while the top 14 is disposed on the base 16. For example, the engagement of the grooves 78A, 78B of the insert 50 with the ridges 22A, 22B of the storage bag shell 10 prevents the insert 50 from moving perpendicular (e.g., moving vertically and/or moving horizontally within the opening 20) relative to the storage bag shell 10 (e.g., moving within the opening 20), while the top 14 is disposed on the base 16.

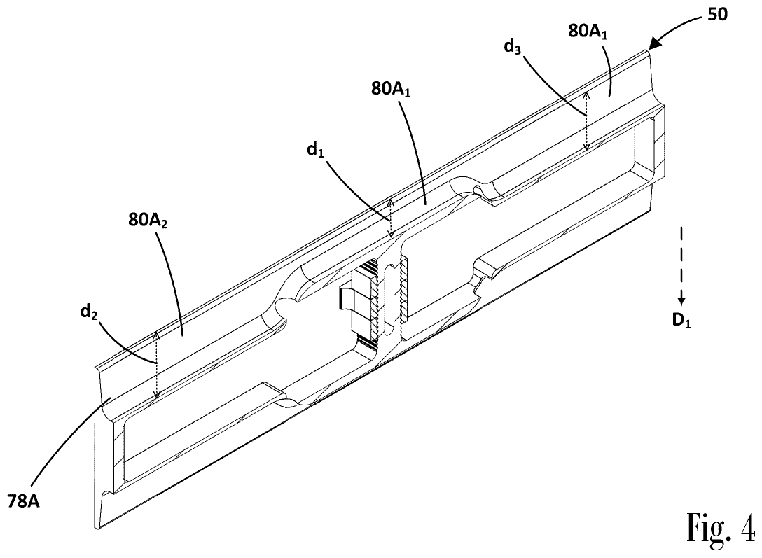

FIG. 4 is a cross-sectional view of the insert 50, according to an embodiment. The cross section in FIG. 4 is along the first groove 78A. For example, the cross section is of the insert 50 along the plane 5-5 indicated in FIG. 1. As shown in FIG. 4, the first groove 78A includes a plurality of groove portions 80A₁, 80A₂, 80A₃. In the illustrated embodiment, the groove portions include a first groove portion 80A₁, a second groove portion 80A₂, and a third groove portion 80A₃. The first groove portion 80A₁ is disposed between the groove portion 80A₂ and the third groove portion 80A₃ (e.g., along a length L of the insert 50). For example, the second groove portion 80A₂ and the third groove portion 80A₃ can be opposite end portions of the first groove 78A. The first groove portion 80A₁ can be referred to as a middle groove portion, the second groove portion 80A₂ can be referred to as the first end groove portion, and the third ridge portion 80A₃ can be referred to as the second end groove portion.

The intermediate portion 72A of the first arm 70A includes the middle groove portion 80A₁ (e.g., the middle groove portion 80A₁ extends through the intermediate portion 72A, the middle groove portion 80A₁ is disposed in the intermediate portion 72A). The first branch 74A of the first arm 70A includes the first end groove portion 80A₂ (e.g., the first end groove portion 80A₂ extends through the first branch 74A, the first end groove portion 80A₂ is disposed in the first branch 74A). The second branch 76A of the first arm 70A includes the second end groove portion 80A₃ (e.g., the second end groove portion 80A₃ extends through the second branch 76A, the second end groove portion 80A₃ is disposed in the second branch 76A).

The groove portions 80A₁, 80A₂, 80A₃ have a plurality of depths d₁, d₂, d₃. For example, the depth of a groove portion is a length that said groove portion extends into the insert 50 (e.g., depth measured in vertical direction D₁). The middle groove portion 80A₁ has a first depth d₁, the first end groove portion 80A₂ has a first depth d₂, and the third end groove portion 80A₃ has a third depth d₃. In the illustrated embodiment, the groove portions 80A₁, 80A₂, 80A₃ have two different depths (e.g., depth d₂ is the same as depth d₃). The end groove portions 80A₂, 80A₃ have same depth (e.g., depth d₂/d₃) that is different from depth d₁ of the middle groove portion 80A₁. The end ridge 80A₂, 80A₃ portions each have a respective depth d₂, d₃ is longer than the depth h₁ of the middle groove portion 80A₁ (i.e., depth d₂>depth d₁ and depth d₃>depth d₁). In another embodiment, the end groove portions 80A₂, 80A₃ portions may each have a respective depth d₂, d₃ that is shorter than the depth d₁ of the middle groove portion 80A₁ (i.e., depth d₂<depth d₁ and depth d₃<depth d₁).

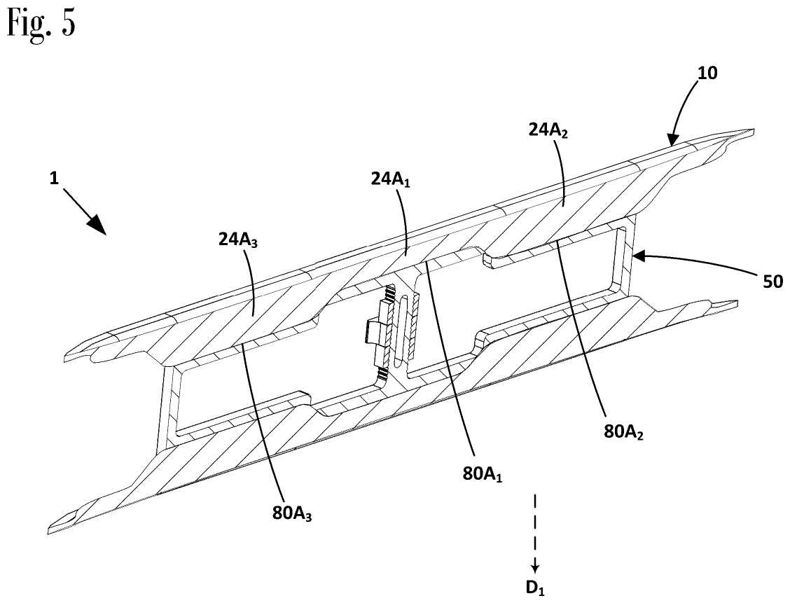

FIG. 5 is a cross-sectional view of the assembly 1 with the temperature probe 92 omitted, according to an embodiment. For example, the cross section is along the plane 5-5 indicated in FIG. 1. The groove portions 80A₁, 80A₂, 80A₃ of the first groove 78A of the insert 50 are configured to receive the corresponding ridge portions 24A₁, 24A₂, 24A₃, of the upper ridge 22A of the storage bag shell 10. In the illustrated embodiment, the middle groove portion 80A₁ of the first groove 78A is configured to receive the middle ridge portion 24A₁, of the upper ridge 22A. The first end groove portion 80A₂ of the first groove 70A is configured to receive the first end ridge portion 24A₁ of the upper ridge 22A. The first end groove portion 80A₂ of the first groove 70A is configured to receive the second end ridge portion 24A₂ of the upper ridge 22A.

The upper ridge 22A of the storage bag shell 10 being disposed in the upper groove 78A can prevent removal of the insert 50 from the opening 20 (e.g., prevent movement of the insert 50 in the opening 20 in the depth direction). The ends of the middle groove portion 80A₁ can contact the first end ridge portion 24A₂ and the second end ridge portion 24A₃ preventing horizontal movement of the insert 50 in the opening 20.

In the illustrated embodiment, the groove 78A includes three groove portions 80A₁, 80A₂, 80A₃. In other embodiments, the groove 78A may include a different number of groove portions 80A₁, 80A₂, 80A₃. In an embodiment, the groove 78A may include a single groove portion 80A₁. In another embodiment, the groove 78A may include just the (end) groove portions 80A₂, 80A₃. For example, the abutment of the ends of the one or more groove portions with one or more ridge portions of storage bag shell can be configured to prevent horizontal movement of the inset 50 in the opening 20.

As shown in FIG. 2, the storage bag shell 10 includes the lower ridge 22B. As shown in FIGS. 3 and 5, the second arm 70B of the insert 50 includes the second groove 78B configured to engage with the lower ridge 22B. As shown in FIGS. 3 and 4, the lower ridge 22B may have features as similarly discussed for the upper ridge 22A, and the second groove 78B may have features as similarly discussed for the first grove 78A. For example, the lower ridge 22B can have a plurality of ridge portions (e.g., a middle ridge portion, a first end ridge portion, and a second end ridge portion) with features as similarly discussed for the upper ridge 22A. For example, the lower groove 78B may have a plurality of groove portions (e.g., a middle grove portion, a first groove portion, and a second groove portion) with features as similarly discussed for the upper groove 78A.

As shown in FIG. 3, the insert 50 may include one or more interconnecting portions 84A, 84B that each connect the arms 70A, 70B to each other (e.g., directly connect one arm to another arm). The interconnecting portions 84A, 84B connect the arms 70A, 70B to each other separate from the coupling portion 60. A first interconnecting portion 84A connects the first arm 70A to the second arm 70B (e.g., directly connects the first arm 70A and the second arm 70B). For example, the first connecting portion 84A connects the first branches 74A, 74B of the first and second arms 70A, 70B to each other (e.g., an end of the first branches 74A opposite to intermediate portion 72A in the first arm 70A to the end of the first branches 74B opposite to the intermediate portion 72B in the second arm 70B). For example, the second connecting portion 84B connects the second branches 76A, 76B of the first and second arms 70A, 70B to each other (e.g., an end of the second branch 76A opposite to intermediate portion 72A in the first arm 70A to an end of the second branch 76B opposite to the intermediate portion 72B in the second arm 70B). As shown in FIG. 3, the first and second interconnecting portions 84A, 84B can provide opposite sides of the insert 50, respectively (e.g., the vertical sides of the insert 50).

In the illustrated embodiment, the insert 50 includes the two grooves 78A, 78B that engage with the two ridges 22A, 22B of the storage bag shell 10. In another embodiment, the storage bag shell 10 may only include a single one of the two ridges 22A, 22B (e.g., does not include the upper ridge 22A, does not include the lower ridge 22B). In such an embodiment, the insert 50 may include a single corresponding groove 70A, 70B. For example, the storage bag shell 10 may include one or more the ridges 22A, 22B and the insert 50 may have one or more of the grooves 78A, 78B.

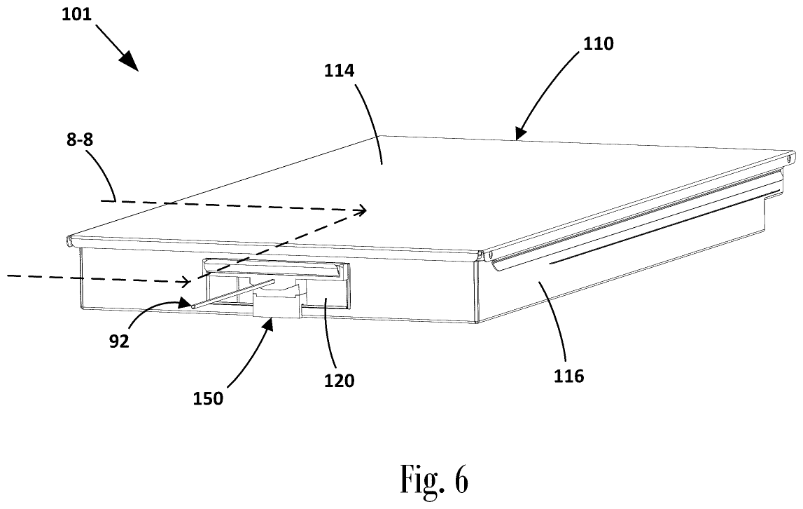

FIG. 6 is a front perspective view of a second embodiment of a storage bag temperature monitoring assembly 100. The assembly 100 includes a storage bag shell 110 and a temperature probe insert 150. The storage bag shell 110 is configured to contain a storage bag 90. The storage bag shell 110 includes an internal volume 112. The storage bag 190 is disposed in the internal volume 112 of the storage bag shell 110. The assembly 100 can be configured to hold the storage bag 90 in the internal volume 112 as similarly discussed for the assembly 1 in FIG. 1.

The storage bag shell 110 is made of a rigid material (e.g., a rigid polymer, or the like). In the illustrated embodiment, the storage bag shell 110 is made of metal. In an embodiment, the storage bag shell 110 may be made as discussed in U.S. Patent Application Publication No. 2023/0023196, which is incorporated by reference. The storage bag shell 10 is configured to provide protection to the storage bag 90. The storage bag shell 10 is a rigid shell configured to protect a storage bag 90 disposed in the internal volume 12.

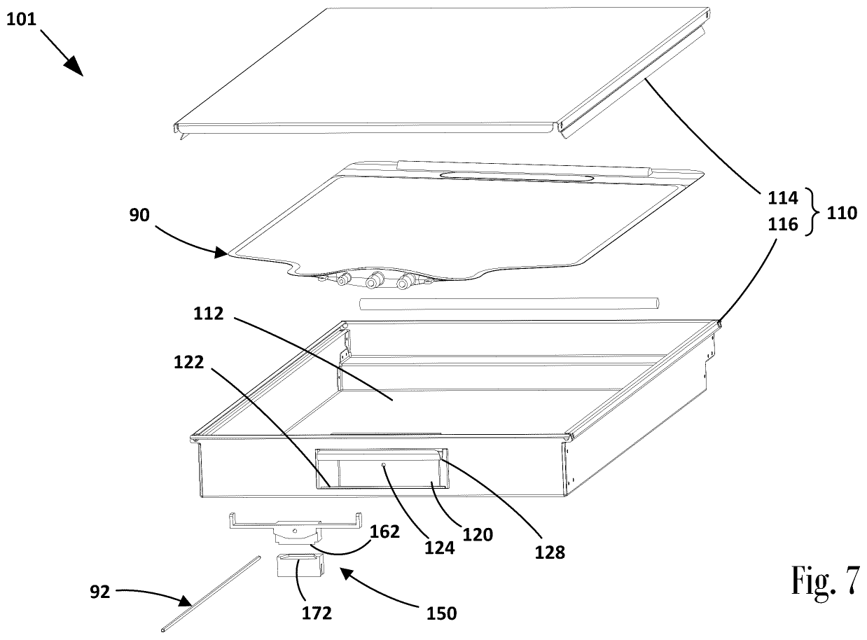

The storage bag shell 110 includes a top 114 and a base 116. The top 114 is disposed on the base 116. The top 14 and the base 116 define the internal volume 112 (shown in FIG. 7) of the storage bag shell 110. For example, the internal volume 112 is formed between the top 114 and the base 116. The storage bag 90 is then trapped without the internal volume 112. In the illustrated embodiment, the top 114 and the base 116 are hinged together (e.g., by one or more pins). In another embodiment, the top 114 may be coupled to the base 116 in a different manner.

FIG. 7 is an exploded view of the assembly 1, according to an embodiment. The storage bag shell 110 includes an opening 120. In the illustrated embodiment, the base 116 of the storage bag shell 110 includes the opening 120. The temperature probe insert 150 is removably disposed in the opening 120 in the storage bag shell 110. The opening 120 includes an inlet passage 126 and an outlet passage 126. The temperature probe insert 150 is disposed in the inlet passage 126 of the opening 120. The opening 120 extends into the internal volume 112 of the storage bag shell 110. For example, the outlet passage 126 of the opening 120 extends through the base 116 to the internal volume 112. The insert 150 is pressure fit into the opening 120. For example, the pressure fit can prevent the insert from being accidently removed from the opening 120. The disposition of the insert 150 in the opening is discussed in more detail below.

The temperature probe insert 150 is configured to align a temperature probe 92 within the storage bag 90, as similarly discussed above with respect to the insert 150. The temperature probe insert 150 includes an aperture 152 for directing the temperature probe 92. The aperture 152 of the insert 150 is configured to direct the temperature probe 192 into a storage bag 90 disposed in the internal volume 112. The insert 150 is also configured to align the aperture 152 with the outlet passage 126 of the opening 120, such that the temperature probe 92 through the aperture 152 is directed towards and through the outlet passage 126. The aperture 152 is discussed in more detail below.

The storage bag shell 110 includes a first ridge 122 that extends into the opening 120 (e.g., extends perpendicular to the axis A_{D2} of the opening 120). The base 116 includes the first ridge 122. The first ridge 122 may be referred to as a bottom ridge.

The storage bag shell 110 includes a handle 128. For example, the base 116 includes the handle 128. The handle 128 is disposed along the opening 120. For example, an end 130 of the handle 128 overlaps with opening (e.g., the inlet passage 124 is at least partially blocked by the end 130 of the handle 128, the end 130 of the handle 128 overlaps with the inlet passage 124 in the depth direction of the opening 120).

The insert 150 includes a coupling portion 160 and a plurality of arms 170A, 170B, 170C that each extend from the coupling portion 160. The aperture 152 extends through the coupling portion 160 of the insert 150. The coupling portion 160 of the insert 150 is disposed in the opening 120 of the storage bag shell 110. The aperture 152 is configured to direct the temperature probe 192 into the internal volume 112. For example, the outlet 152 of the aperture 152 faces towards the outlet passage 126 (e.g., the outlet 152 of the aperture 152 faces towards the internal volume 112). In particular, the aperture 152 is configured to direct a temperature probe 192 into the storage bag 190 disposed in the internal volume 112. The aperture 152 has an outlet 154 that faces towards a central portion of the internal volume 112. For example, the central portion can be the same portion of the internal volume 112 as discussed above for the central portion of the internal volume 12 of in the storage bag shell 10. In an embodiment, a location of the aperture 152 in the coupling portion 160 may be adjustable (e.g., similar to the aperture 52).

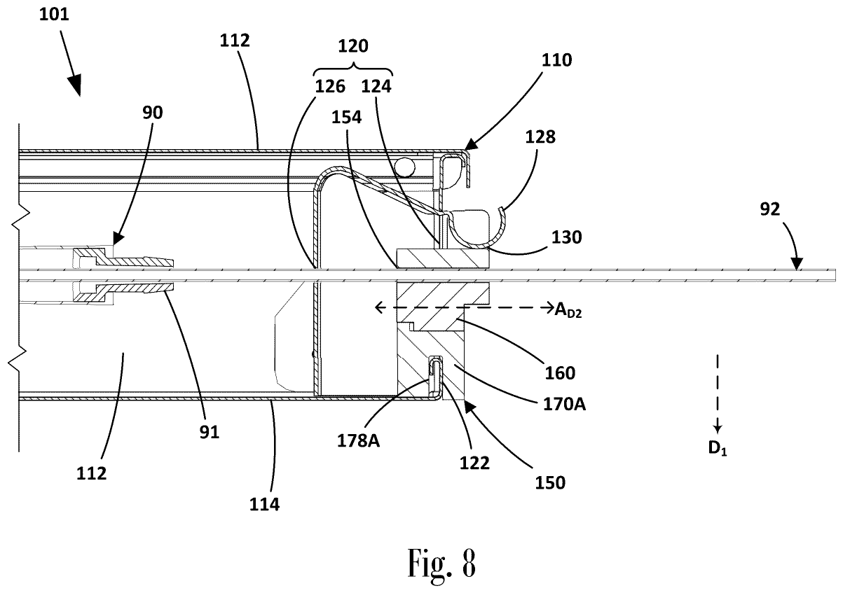

FIG. 8 is a partial cross-sectional view of the assembly 1, according to an embodiment. For example, the cross-sectional view as indicated by 8-8 in FIG. 6. The aperture 152 directs the probe 192 into a port 91 of the storage bag 90. The insert 150 is configured to direct the probe 192 such that an end 194 of the probe 192 is disposed within the storage bag 190. For example, the end 194 of the probe 192 can be configured to be in contact with the fluid within the storage bag 190. The aperture 152 can have a size configured to provide a fit between the aperture 152 and the probe 19. For example, the size can be similar as above for the aperture 52 in the insert 50 in FIGS. 1-5.

The plurality of arms 170A, 170B, 170C are configured to contact the storage bag shell 110 to prevent perpendicular movement of the coupling portion 160 within the opening 120 (e.g., movement perpendicular to opening 120, movement perpendicular to the depth direction D_{D} of the opening 120). For example, the plurality of arms 170A, 170B, 170C contact the storage bag shell 110 preventing movement along a first perpendicular direction D₁ (e.g., in the vertical direction) and along a second perpendicular direction D₂ (e.g., in the horizontal direction).

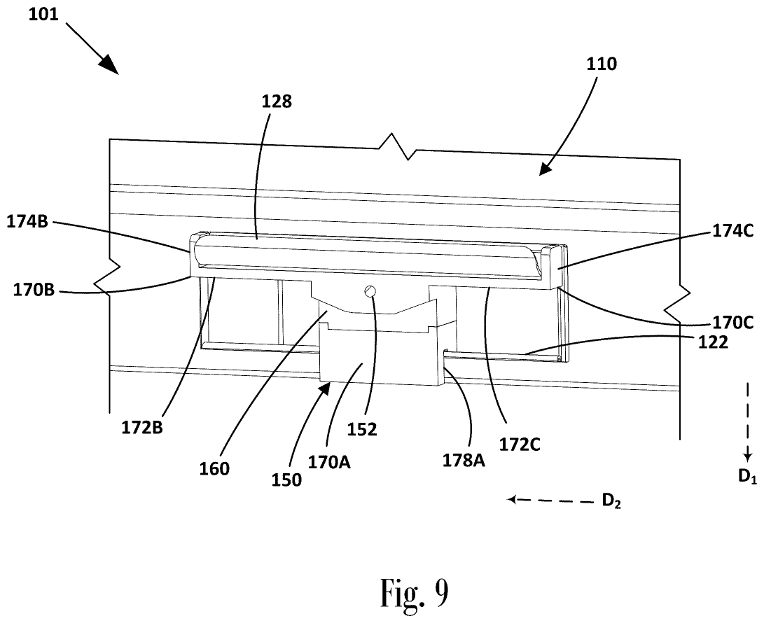

FIG. 9 is a partial front perspective view of the insert 150 disposed in the storage bag shell 110, according to an embodiment. In FIG. 9, the internals of the temperature probe 92 (e.g., thermal couple, connecting wire, and/or the like) are omitted in FIG. 9 for illustrative purposes. In the illustrated embodiment, the plurality of arms 170A, 170B, 170C include a first arm 170A, a second arm 170B, and a third arm 170C. The first arm 170A, the second arm 170B, and the third arm 170C extend from different sides of the coupling portion 160. As shown in FIG. 9, the second arm 170B and the third arm 170C extend from opposite sides of the coupling portion 160A. For example, the first arm 170A, the second arm 170B, and the third arm 170C each extend perpendicular to the aperture 152 (e.g., first arm 170A extending in direction D₁, the second arm 170B extending in direction D₂, third arm 170C extending in a direction opposite to direction D₁).

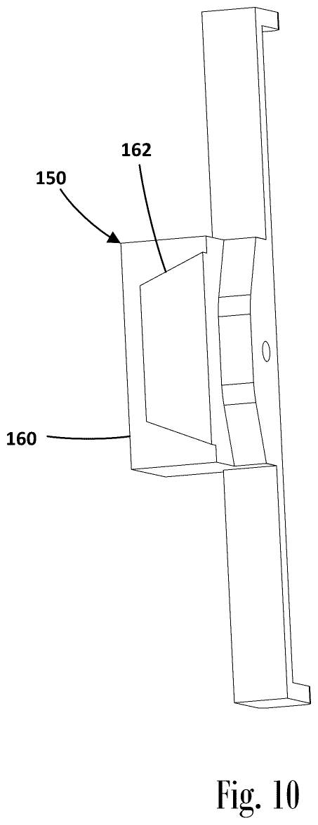

As shown in FIGS. 7 and 9, the first arm 170A can be a separate piece from the coupling portion 160. FIG. 10 shows a bottom perspective view of the insert 130 with the first arm 170A omitted, according to an embodiment. The coupling portion 160 and the first arm 170A include a channel 172 and a projection 162. The channel 172 and projection 162 are configured to couple the coupling portion 160 and the first arm 170A preventing horizontal sliding movement between the coupling portion 160 and the first arm 170A. In the illustrated embodiment, the coupling portion 160 includes the projection 162 and the first arm 170A includes the channel 172. The projection 162 and channel 172 have a configuration that allows for the projection 162 to be slid into the channel 162 (e.g., in a depth direction D_{D}). The coupling between the channel 172 and the projection 162 are then configured to prevent sliding movement along the perpendicular direction (e.g., in direction D₂, in a direction opposite to direction D₂).

The first arm 170A of the insert 150 includes a groove 178A. The groove 178A is formed in a bottom side of the first arm 170A (e.g., in the bottom of the insert 150). The groove 178A is configured to receive the ridge 122 of the storage bag shell 110. The ridge 122 is disposed in the groove 78A. The channel 172A and the groove 178A are disposed on opposite ends (e.g., opposite sides) of the firm arm 170A.

A top of the insert 150 is configured to press against the storage bag shell 110. In the illustrated embodiment, the top of the insert 50 is configured to press against the handle 128 of the storage bag shell 110. For example, the first arm 170A is disposed in the opening 120 on the ridge 122 (e.g., groove 178A placed onto the ridge 122), and then the coupling portion 160 is inserted in the opening 120 with the projection 162 inserted into the channel 172A of the first arm 170A. The top of the insert 150 presses against the handle 128 when inserted into the opening 120. The pressure of the handle 128 on insert 150 is configured to prevent accidental removal of the coupling portion 160 (e.g., accidental removal of the projection 162 from the channel 172A in the first arm 170A. In another embodiment, the insertion of the coupling portion 160 may press the top of the insert 150 against a (top) side of the opening 120 to provide a pressure fit of the insert 150 in the opening 120. The insert 150 is pressure fit in the opening 120 via the first arm 150A. This pressure fit via the first arm 150A is also configured to prevent vertical movement of the insert 150 within the opening 120.

The second arm 170B of the insert 150 includes a first portion 172B and a second portion 172B that extends from the first portion 172B. The third arm 170C of the insert 150 includes a first portion 172C and a second portion 174C that extends from the first portion 172C. In each of the second and third arms 170B, 170C, the first portion 172B, 172C extends from the coupling portion 160 of the insert 150, and the second portion 174B, 174C extends from the first portion. As shown in FIG. 9, the second and third arms 170B, 170C can each have a “L” shape. For example, the angle between the first portion 172B, 172C and the second portion 174B, 174C is 90 degrees or less.

The second portion 174B of the second arm 170B is configured to contact a first side of the handle 128. The second portion 174C of the third arm 170C is configured to contact a second side of the handle 128. The second and third arms 170B, 170C contact opposite sides of the handle 128. The handle 128 is disposed between the second and third arms 170B, 170B of the insert 150. The contact of the arms 170B, 170C on the handle 128 is configured to prevent movement (e.g., horizontal movement) of the insert 150 within the opening 120.

In an embodiment, a knit includes a temperature probe insert (e.g., temperature probe insert 50, temperature probe insert 150) and a temperature probe (e.g., temperature probe 92). In an embodiment, the kit may be used to employ the temperature probe insert to dispose the temperature probe in a storage bag shell (e.g., storage bag shell 10, storage bag shell 110). For example, the kit may be used to employ the storage bag shell to provide a storage bag temperature monitoring assembly (e.g., assembly 1, assembly 101).

The examples disclosed in this application are to be considered in all respects as illustrative and not limitative. In an embodiment, “connects” and “connecting” as described above may refer to “directly connecting”. In an embodiment, “extends” and “extending” as described above may refer to “directing extends” and “directly extending”. The scope of the invention is indicated by the appended claims rather than by the foregoing description; and all changes which come within the meaning and range of equivalency of the claims are intended to be embraced therein.

What is claimed is:

1. A temperature probe insert, comprising:
a coupling portion including aperture that extends through the coupling portion;
a first arm extending from the coupling portion; and
a second arm extending from the coupling portion, the first arm and the second arm extending from different sides of the coupling portion;
wherein the temperature probe insert is configured to be removably disposed in an opening in a storage bag shell, the first arm and the second arm are configured to contact the storage bag shell to prevent movement of the coupling portion in the opening in the storage bag shell, and the aperture configured to direct a temperature probe into a storage bag disposed in an internal volume of the storage bag shell.

2. The temperature probe insert of claim 1, wherein the aperture has a size configured to direct the temperature probe at an angle that disposes an end of the temperature probe in the storage bag.

3. The temperature probe insert of claim 1, wherein a location that the aperture extends through the coupling portion is adjustable.

4. The temperature probe insert of claim 1, wherein the aperture has an outlet configured to face towards a central portion of the internal volume.

5. The temperature probe insert of claim 1, wherein
the first arm includes a first groove, and the first groove is configured to receive a first ridge of the storage bag shell so as to prevent removal of the insert from the opening in the storage bag shell.

6. The temperature probe insert of claim 5, wherein the first groove includes a first groove portion having a first depth and a second groove portion having a second depth different from the first depth, the first groove portion configured to receive a first ridge portion of the first ridge and the second groove portion configured to receive a second ridge portion of the first ridge to prevent movement of the insert within the opening in the storage bag shell.

7. The temperature probe insert of claim 5, wherein the first groove includes a first groove portion, a second groove portion, and a third groove portion having at least two different depths, the first groove portion configured to receive a first ridge portion of the first ridge, the second groove portion configured to receive a second ridge portion of the first ridge, and the third groove portion configured to receive a third ridge portion of the first ridge to prevent movement of the temperature probe insert within the opening in the storage bag shell.

8. The temperature probe insert of claim 5, wherein
the second arm includes a second groove, and the second groove is configured to receive a second ridge of the storage bag shell to prevent removal of the temperature probe insert from the opening in the storage bag shell.

9. The temperature probe insert of claim 1, further comprising:
a third arm slidingly coupled to the coupling portion, the third arm including a groove configured to receive a ridge of the storage bag shell.

10. The temperature probe insert of claim 9, wherein the insert is configured to be pressure fit in the opening via sliding coupling of the third arm with the coupling portion.

11. The temperature probe insert of claim 9, wherein
the first arm includes a first portion extending from the coupling portion and a second portion extending from the first portion,
the second arm includes a first portion extending from the coupling portion and a second portion extending from the first portion of the second arm, and
the temperature probe insert is configured to be disposed in the opening with a handle of the storage bag shell disposed between the first arm and the second arm, so as to prevent movement of the insert within the opening.

12. A storage bag temperature monitoring assembly, comprising:
a storage bag shell including a base and a top disposed on the base to form an internal volume configured contain a storage bag, the storage bag shell including an opening; and
a temperature probe insert removably disposed in the opening in the storage bag shell, the temperature probe insert including:
a coupling portion including aperture that extends through the coupling portion,
a first arm extending from the coupling portion, and
a second arm extending from the coupling portion, the first arm and the second arm extending from different sides of the coupling portion,
wherein the first arm and the second arm contact the storage bag shell preventing movement of the coupling portion in the opening in the storage bag shell, and the aperture is configured to direct a temperature probe into the storage bag disposed in the internal volume of the storage bag shell.

13. The storage bag temperature monitoring assembly of claim 12, wherein positioning of the top and the base of the storage bag shell prevents removal of the temperature probe insert from the opening in the storage bag shell without moving of at least one of the top and the base of the storage bag shell.

14. The storage bag temperature monitoring assembly of claim 12, further comprising:
a temperature probe disposed in the aperture of the temperature probe insert.

15. The storage bag temperature monitoring assembly of claim 12, wherein a clearance fit is formed between the temperature probe and the aperture in the coupling portion.

16. The storage bag temperature monitoring assembly of claim 12, wherein
the storage bag shell includes a first ridge having a first ridge portion, a second ridge portion, and a third ridge portion, and
the temperature probe insert includes a first groove having a first groove portion, a second groove portion, and a third groove portion having at least two different depths, the first ridge portion disposed in the first groove portion, a second ridge portion disposed in the second ridge portion, and the third ridge portion disposed in the third groove portion so as to prevent movement of the insert within the opening in the storage bag shell.

17. The storage bag temperature monitoring assembly of claim 12, wherein the temperature probe insert is friction fit in the opening in the storage bag shell.

18. A kit comprising:
a temperature probe insert comprising:
a coupling portion including aperture that extends through the coupling portion;
a first arm extending from the coupling portion. and
a second arm extending from the coupling portion, the first arm and the second arm extending from different sides of the coupling portion,
wherein the temperature probe insert is configured to be removably disposed in an opening in a storage bag shell, the first arm and the second arm are configured to contact the storage bag shell to prevent movement of the coupling portion in the opening in the storage bag shell; and
a temperature probe configured to be disposed in the aperture of the temperature probe insert, the aperture configured to direct the temperature probe into a storage bag disposed in an internal volume of the storage bag shell and the temperature probe configured to detect a temperature of fluid in the storage bag.

19. The kit of claim 18, wherein the aperture has a size configured to form a clearance fit between the aperture and the temperature probe.

20. The kit of claim 18, wherein the aperture has an outlet configured to direct the temperature probe towards a central portion of the internal volume.
